(19) United States
(12) Patent Application Publication
(10) Pub. No.: US 2015/0352140 A1
(43) Pub. Date: Dec. 10, 2015

(54) BIOMASS-BASED DIETARY SUPPLEMENT

(71) Applicant: API Intellectual Property Holdings, LLC, Atlanta, GA (US)

(72) Inventor: Vesa PYLKKANEN, Atlanta, GA (US)

(21) Appl. No.: 14/732,221

(22) Filed: Jun. 5, 2015

Related U.S. Application Data
(60) Provisional application No. 62/009,178, filed on Jun. 7, 2014.

(51) Int. Cl. A61K 31/717; A23L 1/29; A23L 1/308; A23K 1/16; A23L 1/304; A23L 1/302; A23L 1/00; A61K 31/19; A23L 1/226

(52) U.S. Cl. CPC A61K 31/717; A61K 31/19; A23L 1/293; A23L 1/3082; A23L 1/226; A23L 1/304; A23L 1/302; A23L 1/0029; A23L 1/0017; A23L 1/0035; A23K 1/16; A23V 2002/00

(57) ABSTRACT

A biomass-derived dietary supplement comprising an acetate salt, a hemicellulose-derived sweetener, and cellulose is provided. The formulation may originate from treatment of biomass, by converting acetic acid (from biomass) to an acetate salt and combining it with a hemicellulose-based sweetener such as xylitol and inert, non-digestive cellulose. This is formed into a powder, crystal, pill or capsule to be delivered orally as a dietary supplement. Supplemental minerals and vitamins may be added. A process for producing a biomass-derived dietary supplement is also disclosed.

### PRIORITY DATA

This patent application is a non-provisional patent application claiming priority to U.S. Provisional Patent App. No. 62/009,178, filed on Jun. 7, 2014, which is hereby incorporated by reference herein.

### FIELD

The present invention relates, in general, to an oral dietary supplement preparation and, in particular, to manufacture and composition of dietary supplements produced from biomass.

### BACKGROUND

Obesity and diabetes contribute to long-term illnesses and related healthcare costs. The occurrence has increased with sedentary lifestyles and propensity of consuming prepared meals. While these conditions are manageable or in some cases preventable by lifestyle changes, the proliferation of obesity is expected to top 50% of the population in some industrial countries during this century.

Governments promote more nutritious meal plans to advocate healthy diets. USDA's food pyramid and plate method have been used to portion perfect meals. These recommendations are not followed by the vast majority of the people. One reason is that the hunger quenches only after eating and it is difficult to judge how much is needed. When the human brain receives a signal from the satisfying condition, it is too late.

Recently, researchers at Imperial College in London have discovered that large amounts of acetate from biomass reduces appetite in animals. Prof. Frost et al. has shown how acetate reduces appetite (see, for example, Frost et al., “The short-chain fatty acid acetate reduces appetite via a central homeostatic mechanism,” NATURE COMMUNICATIONS, Vol: 5 (2014). This acetate sends a signal to the brain to reduce huger feelings.

Lignocellulosic biomass is the most abundant renewable material on the planet and has long been recognized as a potential feedstock for producing chemicals, fuels, and materials. Lignocellulosic biomass normally comprises primarily cellulose, hemicellulose, and lignin. Cellulose and hemicellulose are natural polymers of sugars, and lignin is an aromatic/aliphatic hydrocarbon polymer reinforcing the entire biomass network.

Retsina et al. (U.S. Pat. Nos. 8,518,672, 8,518,213, 8,211,680, 8,679,364, and 8,685,685, which are incorporated by reference herein) teach separation and conversion of acetyl groups in hemicellulose to acetates from wood hemicellulose. American Process Inc. has constructed a plant for biomass-based supply of potassium acetate.

Presently, Nelson et al. (U.S. patent application Ser. Nos. 14/092,906, 14/092,908, and 14/092,910, which are incorporated by reference herein) has taught an application for manufacture of cellulose nanocrystals (CNC). CNC is derived from the crystalline portion of cellulose, which is the base unit for plant fiber. Microcrystalline cellulose is used for non-active filler for various medical capsules and pills, for example.

Plant hemicellulose-based sweeteners have been developed such as xylitol. Xylitol is a five-carbon sugar alcohol that can be found in nature in small quantities. The hemicellulosic xylan is recovered from pulping spent liquor and converted to monomers by hydrolysis. The xylose sugar is further converted to xylitol by fermentation or chemical reaction. Xylitol is a sweetener, but it is not processed to energy in the human body. It has attracted global attention because of its sweetening power similar to that of sucrose, but provides much fewer calories. Xylitol is also known to be metabolized through insulin-independent pathways in humans and therefore can be used as sugar substitute for diabetics.

Currently, xylitol is industrially produced by chemical hydrogenation of xylose-containing hemicellulosic hydrolysate in the presence of a metal catalyst under high temperature and pressure. However, this chemical process is costly and energy-intensive; in addition, there is also a need for a complex purification and separation process. In order to produce xylitol in a more environmental-friendly manner, research has been conducted on alternative strategies that utilize microorganisms for conversion of xylose-to-xylitol from hemicelluloses (Prakasham et al., “Current trends in biotechnological production of xylitol and future prospects,” Curr. Trends Biotechnol. Pharm. 3, 8-36, 2009; Granström, et al., “A rare sugar xylitol. Part I: the biochemistry and biosynthesis of xylitol,” Appl. Microbiol. Biotechnol. 74, 277-281, 2007).

### SUMMARY

In some variations, the invention provides a biomass-derived dietary composition comprising an acetate salt, a hemicellulose-derived sweetener, and cellulose.

The acetate salt may be selected from the group consisting of sodium acetate, potassium acetate, magnesium acetate, calcium acetate, ammonium acetate, and combinations thereof

In some embodiments, the hemicellulose-derived sweetener comprises or consists essentially of xylitol.

The cellulose may be in the form of purified microcellulose, purified nanocellulose, or a combination thereof

The composition may further include other naturally occurring materials or derivatives from biomass. Also, the composition may further include minerals (which may or may not be derived from biomass) and/or vitamins.

The composition may be in the form of a paste, powder, crystal, pill, or capsule, for example. In some embodiments, the composition is present in a pill or capsule that is coated with a natural polymer capable of releasing the composition over a period of time, after consumption by a human or other animal.

Other variations of the invention provide a process for producing a dietary composition, the process comprising:
(a) extracting xylose oligomers and acetic acid from a feedstock comprising lignocellulosic biomass, thereby generating cellulose-rich solids;
(b) hydrolyzing the xylose oligomers using an acid catalyst or enzymes to generate xylose;
(c) converting the xylose to xylitol using a chemical catalyst or a microorganism;
(d) converting the acetic acid to an acetate salt by reacting the acetic acid with a base;
(e) obtaining purified cellulose from the cellulose-rich solids;
(f) combining the xylitol, the acetate salt, and the purified cellulose to form a mixture; and
(g) recovering the mixture as a dietary composition.

In some embodiments, the feedstock is selected from hardwoods or agricultural residues.

Various extraction technologies may be utilized. In some embodiments, step (a) utilizes steam and/or hot water, optionally with an extraction catalyst. An exemplary extraction catalyst is acetic acid, which may be then converted (at least in part) to an acetate salt. In other embodiments, step (a) utilizes an extraction catalyst, a solvent for lignin, and water. In certain embodiments, step (b) utilizes a sulfur-containing acid catalyst which is optionally derived from step (a). For example, the sulfur-containing acid catalyst could be sulfur dioxide used in step (a) and which remains in solution for step (b). As another example, the sulfur-containing acid catalyst could be lignosulfonic acid created in step (a).

Various bases may be used in step (d). The acetate salt may be selected from the group consisting of sodium acetate, potassium acetate, magnesium acetate, calcium acetate, ammonium acetate, and combinations thereof.

In some embodiments, depending on the chemistry of the initial extraction, the process further comprises bleaching the cellulose-rich solids to reduce lignin content. In some embodiments, the process further comprises refining the cellulose-rich solids to reduce cellulose particle size. Bleaching, if performed, may be done before or after refining, if desired. The purified cellulose may include microcrystalline cellulose and/or nanocrystalline cellulose. In certain embodiments, the cellulose is sufficiently low in lignin and particle size from extraction (fractionation) that little or no refining or bleaching is necessary.

The dietary composition may be formed into a paste, powder, crystal, pill, or capsule. In some embodiments, the process further comprises coating the dietary composition with a natural polymer. After consumption of the dietary composition by a human or other animal, the natural polymer slowly releases the dietary composition over a period of time, such as hours or days.

### DETAILED DESCRIPTION OF SOME EMBODIMENTS

This description will enable one skilled in the art to make and use the invention, and it describes several embodiments, adaptations, variations, alternatives, and uses of the invention. These and other embodiments, features, and advantages of the present invention will become more apparent to those skilled in the art when taken with reference to the following detailed description of the invention in conjunction with any accompanying drawings.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs. All composition numbers and ranges based on percentages are weight percentages, unless indicated otherwise. All ranges of numbers or conditions are meant to encompass any specific value contained within the range, rounded to any suitable decimal point.

Unless otherwise indicated, all numbers expressing parameters, reaction conditions, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” limits the scope of a claim to the specified elements or method steps, plus those that do not materially affect the basis and novel characteristic(s) of the claimed subject matter.

With respect to the terms “comprising,” “consisting of,” and “consisting essentially of,” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of”

The present inventor has discovered that the three major components in biomass can be converted to an effective dietary composition (e.g., supplement) that quenches hunger in humans or other animals. After separation of acetyl groups from the biomass and combining the resulting acetic acid with a soluble base, the resulting acetate salt can be then provided in a sweet pill form. The pill may include a hemicellulose-based sweetener and a non-digestive cellulose.

Certain exemplary embodiments of the invention will now be described. These embodiments are not intended to limit the scope of the invention as claimed. The order of steps may be varied, some steps may be omitted, and/or other steps may be added. Reference herein to first step, second step, etc. is for illustration purposes only.

In some variations, the present invention consists of a formulation originating from treatment of biomass, separation of acetic acid and conversion to an acetate salt, and combination of the acetate salt with a hemicellulose-based sweetener as well as inert cellulose. This formulation may be in the form of a powder, crystals, a pill, or a capsule, to be delivered orally as a dietary supplement. Naturally occurring materials from biomass and supplemental minerals and vitamins may optionally be present or added.

In some embodiments, a formulation of dietary supplement is a mixture of biomass-derived potassium acetate, cellulose, and hemicellulose-derived sweetener. The formulation may reduce appetite for dieters, for example. The formulation targets better acceptance within public by using all natural ingredients.

A first ingredient comprises acetic acid from treatment of biomass, such as wood chips (preferably hardwoods), agricultural residues, grasses, corn or other grains. Hemicelluloses, which contain acetyl groups, may be cleaved in thermal or chemical treatment. Hemicellulose extraction may be accomplished by treatment with steam or hot water. The liberation of acetyl groups may be increased by a secondary chemical or heat treatment of the extract liquor.

An alkaline component is added to the acetic acid, to form an acetate salt. Exemplary acetate salts include sodium, potassium, magnesium, calcium, and ammonia salts. The resulting acetate salt may contain a small amount of incidental or deliberate additives, such as formic acid and lactic acid. Purification of the acetate can be performed using ion exchange, activated carbon, filtration, or another selective process. The purification may combine several steps to achieve removal of unwanted components. The acetate is preferably dried or crystallized to a solid form.

A second ingredient comprises a hemicellulosic sweetener, which may be derived from the hemicellulosic xylose released during the acetyl group deliberation. Xylose may be also derived from pulping spent liquor. Xylose may be converted to xylitol by chemical catalysis or by fermentation. A chromatographic method may be applied to purify xylitol. Finally, xylitol is preferably crystallized to a solid form.

A third ingredient comprises a cellulose filler, which may be derived from the cellulose remaining after hemicellulose and acetyl group liberation. Cellulose may be also derived from another pulping process. Cellulose may be purified by means of bleaching to remove lignin. The white (bleached) cellulose may refined or homogenized or otherwise reduced to a size which is suitable for human digestion. The final cellulose product is typically sized to less than a micron (e.g., microcellulose or nanocellulose). The cellulose may contain some moisture before adding the other ingredients.

Wide ranges of component concentrations are possible. In some embodiments, cellulose content may be from about 50 wt % to about 99 wt %, such as about 88 wt % to about 97 wt % (all on a dry basis). In some embodiments, xylitol (or other hemicellulosic sweetener) concentration may be from about 0.1 wt % to about 10 wt %, such as about 1 wt % to about 5 wt %. In some embodiments, acetate salt concentration may be from about 0.5 wt % to about 10 wt %, such as about 1 wt % to about 5 wt %. In any of these embodiments, other components (additives, fillers, minerals, vitamins, impurities, and so on) may be present, such as collectively from about 0.5 wt % to about 10 wt %, or about 1 wt % to about 5 wt %.

In some embodiments, all the ingredients are mixed to form a uniform paste. This paste may be extruded to form uniform shapes and compressed to final form. Additives, if any, such as minerals, vitamins, sugars, other sweeteners, or sweetening modifiers, may be introduced as well. In certain embodiments, the pill or capsule may be coated with natural a polymer for slow release of the acetate salt and/or hemicellulosic sweetener. A “natural polymer” for this disclosure means a polymer that is naturally occurring (e.g., a starch-based polymer, a protein, a lipid, or a polysaccharide). Examples include corn starch, chitosan, carrageenan, xanthan gum, α-tocopherol, and combinations thereof.

In some variations, the invention provides a biomass-derived dietary composition comprising an acetate salt, a hemicellulose-derived sweetener, and cellulose, which each may be obtained from any source and then combined.

The acetate salt may be selected from the group consisting of sodium acetate, potassium acetate, magnesium acetate, calcium acetate, ammonium acetate, and combinations thereof In some embodiments, the hemicellulose-derived sweetener comprises or consists essentially of xylitol. The cellulose may be in the form of purified microcellulose, purified nanocellulose, or a combination thereof.

The composition may further include other naturally occurring materials or derivatives from biomass. Also, the composition may further include minerals (which may or may not be derived from biomass) and/or vitamins. In principle, certain vitamins could be obtained from biomass by fermenting biomass-derived sugars to vitamins (e.g., vitamin C).

The composition may be in the form of a paste, powder, crystal, pill, or capsule, for example. In some embodiments, the composition is present in a pill or capsule that is coated with a natural polymer capable of releasing the composition over a period of time, after consumption by a human or other animal.

Suitable xylitol-producing microorganisms are also known in the art. For example, see Huang, et al., “Development of a yeast strain for xylitol production without hydrolysate detoxification as part of the integration of co-product generation within the lignocellulosic ethanol process”, Bioresource Technology 102 (2011) 3322-3329; Latif et al., “Production of ethanol and xylitol from corn cobs by yeasts,” Bioresource Technology 77 (2001) 57-63; and Rao et al., “Xylitol production by Candida sp.: parameter optimization using Taguchi approach,” Process Biochemistry 39 (2004) 951-956, each of which is hereby incorporated by reference.

Chemical catalysis of xylose to xylitol is known. For example, a sponge nickel catalyst (commonly referred to as Raney Ni catalyst) may be employed. See, for example, Mikkola et al., “Hydrogenation of xylose to xylitol on sponge nickel catalyst—a study of the process and catalyst deactivation kinetics,” Brazilian Journal of Chemical Engineering, vol. 20 no. 3 Sao Paulo July/September 2003, which is hereby incorporated by reference herein.

Other sugar alcohols derived from hemicellulosic sugars may be included as hemicellulosic sweeteners (e.g., mannitol). Another hemicellulosic sweetener that may be employed is xylose itself. Xylose is not as sweet as xylitol, which is about as sweet as sucrose. In some embodiments, a relatively small amount of glucose, fructose, or sucrose is included in the dietary supplement for enhancement of sweetness. These sugars may also be obtained from the same source of biomass. For example, glucose may be obtained from enzymatic hydrolysis of cellulose, followed by purification (e.g., electrodialysis). Or sucrose may be obtained from sugarcane while the associated sugarcane bagasse or straw is processed to produce cellulose, acetate salt, and xylitol. Erythritol and other polyols (e.g., maltitol, sorbitol, or mannitol) may also be included.

Other variations of the invention provide a process for producing a dietary composition, the process comprising:
(a) extracting xylose oligomers and acetic acid from a feedstock comprising lignocellulosic biomass, thereby generating cellulose-rich solids;
(b) hydrolyzing the xylose oligomers using an acid catalyst or enzymes to generate xylose;
(c) converting the xylose to xylitol using a chemical catalyst or a microorganism;
(d) converting the acetic acid to an acetate salt by reacting the acetic acid with a base;
(e) obtaining purified cellulose from the cellulose-rich solids;
(f) combining the xylitol, the acetate salt, and the purified cellulose to form a mixture; and
(g) recovering the mixture as a dietary composition.

In some embodiments, the feedstock is selected from hardwoods or agricultural residues.

Various extraction technologies may be utilized. In some embodiments, step (a) utilizes steam and/or hot water, optionally with an extraction catalyst. An exemplary extraction catalyst is acetic acid, which may be then converted (at least in part) to an acetate salt. In other embodiments, step (a) utilizes an extraction catalyst, a solvent for lignin, and water. In certain embodiments, step (b) utilizes a sulfur-containing acid catalyst which is optionally derived from step (a). For example, the sulfur-containing acid catalyst could be sulfur dioxide used in step (a) and which remains in solution for step (b). As another example, the sulfur-containing acid catalyst could be lignosulfonic acid created in step (a).

Various bases may be used in step (d). The acetate salt may be selected from the group consisting of sodium acetate, potassium acetate, magnesium acetate, calcium acetate, ammonium acetate, and combinations thereof

In some embodiments, depending on the chemistry of the initial extraction, the process further comprises bleaching the cellulose-rich solids to reduce lignin content. In some embodiments, the process further comprises refining the cellulose-rich solids to reduce cellulose particle size. Bleaching, if performed, may be done before or after refining, if desired. The purified cellulose may include microcrystalline cellulose and/or nanocrystalline cellulose. In certain embodiments, the cellulose is sufficiently low in lignin and particle size from extraction (fractionation) that little or no refining or bleaching is necessary.

The dietary composition may be formed into a paste, powder, crystal, pill, or capsule. In some embodiments, the process further comprises coating the dietary composition with a natural polymer. After consumption of the dietary composition by a human or other animal, the natural polymer slowly releases the dietary composition over a period of time, such as about 1-24 hours or about 1-10 days.

The biomass feedstock may be selected from hardwoods, softwoods, forest residues, industrial wastes, pulp and paper wastes, consumer wastes, or combinations thereof Some embodiments utilize agricultural residues, which include lignocellulosic biomass associated with food crops, annual grasses, energy crops, or other annually renewable feedstocks. Exemplary agricultural residues include, but are not limited to, corn stover, corn fiber, wheat straw, sugarcane bagasse, sugarcane straw, rice straw, oat straw, barley straw, miscanthus, energy cane straw/residue, or combinations thereof

As used herein, “lignocellulosic biomass” means any material containing cellulose and lignin. Lignocellulosic biomass may also contain hemicellulose. Mixtures of one or more types of biomass can be used. In some embodiments, the biomass feedstock comprises both a lignocellulosic component (such as one described above) in addition to a sucrose-containing component (e.g., sugarcane or energy cane) and/or a starch component (e.g., corn, wheat, rice, etc.).

Various moisture levels may be associated with the starting biomass. The biomass feedstock need not be, but may be, relatively dry. In general, the biomass is in the form of a particulate or chip, but particle size is not critical in this invention.

In some embodiments, a portion of cellulose-rich solids (obtained from extraction of starting biomass) is utilized as pulp for production of a material (such as nanocellulose), a pellet, a pulp product, or a consumer product. A portion of the cellulose-rich solids may also be enzymatically hydrolyzed to produce glucose. Alternatively, or additionally, a portion of cellulose-rich solids (e.g., high-lignin cellulose and/or unrefined cellulose) may be combusted to produce energy.

Reaction conditions and operation sequences may vary widely. In some embodiments, the process is a variation of the AVAP® process technology which is commonly owned with the assignee of this patent application. In some embodiments, the process is a variation of the Green Power+® process technology which is commonly owned with the assignee of this patent application.

Any stream generated by the disclosed processes may be partially or completed recovered, purified or further treated, analyzed (including on-line or off-line analysis), and/or marketed or sold.

Apparatus may be configured for carrying out the disclosed processes using chemical-engineering principles known in the art as well as principles disclosed in commonly owned patents and patent applications, cited above and incorporated by reference herein.

### Example

Northern hardwood liquid extract from a masonite steam explosion process was collected after steaming, refining, and washing the residual wood pulp. The extract consisted of approximately 1.0 wt % of dissolved solids and 0.1 wt % of suspended solids. After hydrolyzing with 3 wt % sulfuric acid for 1 hour at 120° C., the average acetic acid concentration increased tenfold from 0.04 mg/ml to 0.4 mg/ml. The extract was pumped through a nanomembrane, which concentrated the dissolved solids, but allowed smaller molecules to pass to permeate. About 250 gallons of permeate was neutralized first with sodium hydroxide and then potassium hydroxide. The permeate was pumped at 400 psig through a tight RO membrane to obtain 0.89% potassium acetate solution.

The acetate solution was concentrated to 50 wt %. The concentrate developed a precipitate. The precipitate was analyzed for the metal content. The metals are believed to have originated from the wood, chemicals and equipment used in the preparation of the acetate. The inorganic metal content of the precipitate was measured as follows (all percentages are in weight basis): Ca=7.8%; Cr=2.4%; Cu=0.3%; Fe=17.0%; K=17.1%; Mg=1.5%; Mn=0.4%; Na=0.5%; Ni=1.0%; P=0.8%; S=3.4%; Si=0.4%; and Zn=1.0%.

The concentrated acetate was passed through an activated carbon column to remove color. A clear purified acetate was obtained with an organic acid profile as follows: potassium acetate=50.3 wt %; potassium formate=8.1%; and potassium lactate=0.2 wt %. This purified acetate is suitable as an acetate salt component as described herein. The purified acetate may be combined with xylitol and purified cellulose, to form a dietary supplement.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A biomass-derived dietary composition comprising an acetate salt, a hemicellulose-derived sweetener, and cellulose.

2. The biomass-derived dietary composition of claim 1, wherein said acetate salt is selected from the group consisting of sodium acetate, potassium acetate, magnesium acetate, calcium acetate, ammonium acetate, and combinations thereof.

3. The biomass-derived dietary composition of claim 1, wherein said hemicellulose-derived sweetener comprises xylitol.

4. The biomass-derived dietary composition of claim 1, wherein said hemicellulose-derived sweetener consists essentially of xylitol.

5. The biomass-derived dietary composition of claim 1, wherein said cellulose is in the form of purified microcellulose.

6. The biomass-derived dietary composition of claim 1, wherein said cellulose is in the form of purified nanocellulose.

7. The biomass-derived dietary composition of claim 1, wherein said composition further comprises other naturally occurring materials from biomass.

8. The biomass-derived dietary supplement of claim 1, wherein said composition further comprises minerals and/or vitamins.

9. The biomass-derived dietary composition of claim 1, wherein said composition is in the form of a paste, powder, crystal, pill, or capsule.

10. The biomass-derived dietary composition of claim 1, wherein said composition is present in a pill or capsule that is coated with a natural polymer capable of releasing said composition over a period of time, after consumption by a human or other animal.

11. A process for producing a dietary composition, said process comprising:
(a) extracting xylose oligomers and acetic acid from a feedstock comprising lignocellulosic biomass, thereby generating cellulose-rich solids;
(b) hydrolyzing said xylose oligomers using an acid catalyst or enzymes to generate xylose;
(c) converting said xylose to xylitol using a chemical catalyst or a microorganism;
(d) converting said acetic acid to an acetate salt by reacting said acetic acid with a base;
(e) obtaining purified cellulose from said cellulose-rich solids;
(f) combining said xylitol, said acetate salt, and said purified cellulose to form a mixture; and
(g) recovering said mixture as a dietary composition.

12. The process of claim 11, wherein said feedstock is selected from hardwoods or agricultural residues.

13. The process of claim 11, wherein step (a) utilizes steam and/or hot water, optionally with an extraction catalyst.

14. The process of claim 11, wherein step (a) utilizes an extraction catalyst, a solvent for lignin, and water.

15. The process of claim 11, wherein step (b) utilizes a sulfur-containing acid catalyst which is optionally derived from step (a).

16. The process of claim 11, wherein said acetate salt is selected from the group consisting of sodium acetate, potassium acetate, magnesium acetate, calcium acetate, ammonium acetate, and combinations thereof.

17. The process of claim 11, said process further comprising bleaching said cellulose-rich solids to reduce lignin content.

18. The process of claim 11, wherein said purified cellulose includes microcrystalline cellulose and/or nanocrystalline cellulose.

19. The process of claim 11, wherein said dietary composition is formed into a paste, powder, crystal, pill, or capsule.

20. The process of claim 19, said process further comprising coating said dietary composition with a natural polymer, wherein after consumption of said dietary composition by a human or other animal, said natural polymer releases said dietary composition over a period of time.
